(19) United States
(12) Patent Application Publication
(10) Pub. No.: US 2009/0198099 A1
(43) Pub. Date: Aug. 6, 2009

(54) In vivo imaging system

(76) Inventor: Stephen R. Myers, Columbus, OH (US)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/012,739

(22) Filed: Feb. 5, 2008

(51) Int. Cl. A61B 1/05

(52) U.S. Cl. 600/109

(57) ABSTRACT

An in vivo imaging system for viewing the interior of an organism includes a camera mounted to a sled body that is placed in the interior of the organism. The camera includes a lens, and a dome covers the lens. An image display device is provided to display images received from the camera. A cable communicates images to the image display device according to a viewing angle of the lens. A magnetic source body placed on the exterior of the organism magnetically attracts the sled body to hold the sled body in place in the interior of the organism.

### BACKGROUND OF THE INVENTION

Over the last few decades, surgery at in vivo sites (herein to be understood as a location inside an organism) has been revolutionized by the use of the laparascope. The laparascope is an imaging device that has led to the development of minimally invasive surgery procedures because it can transmit images from an in vivo site, permitting a surgeon to view and operate at the in vivo site without having to directly view the site by cutting the patient open. Instead, several small incisions are made in the skin, and specialized tubes or ports are placed therein to accept passage of the laparoscope and other long narrow instruments employed in the operation being performed.

In complex operations, it is common to have multiple ports for the passage of the laparoscope. This ensures that different viewing angles can be recorded by the laparoscope to help visualize the operations taking place in vivo. While such extra ports are useful, they result in greater invasion of the patient's body, and some views of the patient's interior might still be difficult to obtain.

Rarely is the laparoscope inserted into the in vivo site and then left alone. Rather, the laparoscope is constantly manipulated to obtain a new view or readjust the desired view, for instance when the laparoscope is jostled out of the desired position. The laparoscope is sometimes in the way of desired operation procedures and must often be manipulated to get out of the way of other surgical instruments.

Thus, a need exists in the art for better methods and devices for placing an image device at an in vivo site to transmit images to an image display device so that a surgeon or other caregiver can view a patient's interior.

### SUMMARY OF THE INVENTION

This invention generally provides an in vivo imaging system for viewing the interior of an organism. The system includes a camera mounted to a sled body that is placed in the interior of the organism. The camera includes a lens, and a dome covers the lens. An image display device is provided to display images received from the camera. A cable provides power to the camera and communicates images to the image display device according to a viewing angle of the lens. A magnetic source body placed on the exterior of the body magnetically attracts the sled body to hold the sled body in place in the interior of the organism.

This invention also provides a method of taking images at a in vivo site. In accordance with the method, a camera assembly is positioned at an in vivo site, the camera assembly including a sled body, a camera having a lens, and a dome covering the lens. The camera is mounted to the sled body and receives images in accordance with a viewing angle of the lens. A magnetic source body is positioned at an external, non-in vivo site, and is aligned with the camera assembly to attract the sled body and thereby secure it at the in vivo site. Images received by the camera are transmitted to an image display device for displaying the images.

In a particular embodiment, the camera is mounted stationary to the sled body, and the view taken in by the camera can be altered by moving the magnetic source body that attracts the sled body. In another embodiment, the camera is mounted to the sled body so as to be movable relative to the sled body, and different views can be taken in by the camera by moving the camera relative to the sled body.

### DESCRIPTION OF THE DRAWINGS

FIG. 1 is a general representation of the various elements of an in vivo imaging system of this invention.

FIG. 2 is a top plan view of the camera assembly of the imaging system;

FIG. 3 is a side elevation view of the camera assembly;

FIG. 4 is a side elevation view of the interaction of the camera assembly and the magnetic source body, showing how the camera assembly is retained in position at an in vivo site, particularly the abdomen;

FIG. 5 is a side elevation view as in FIG. 4, but showing the magnetic source body manipulated to a different position to change the viewing angle of the camera at the camera assembly;

FIG. 6 is a top plan view of an alternative embodiment of a magnetic source body, having a different grip element;

FIG. 7 is a cross section of a cable embodiment;

FIG. 8 is a side elevation view of an alternative camera assembly of the imaging system; and

FIG. 9 is a top plan view of the alternative camera assembly of FIG. 8.

### DESCRIPTION OF THE INVENTION

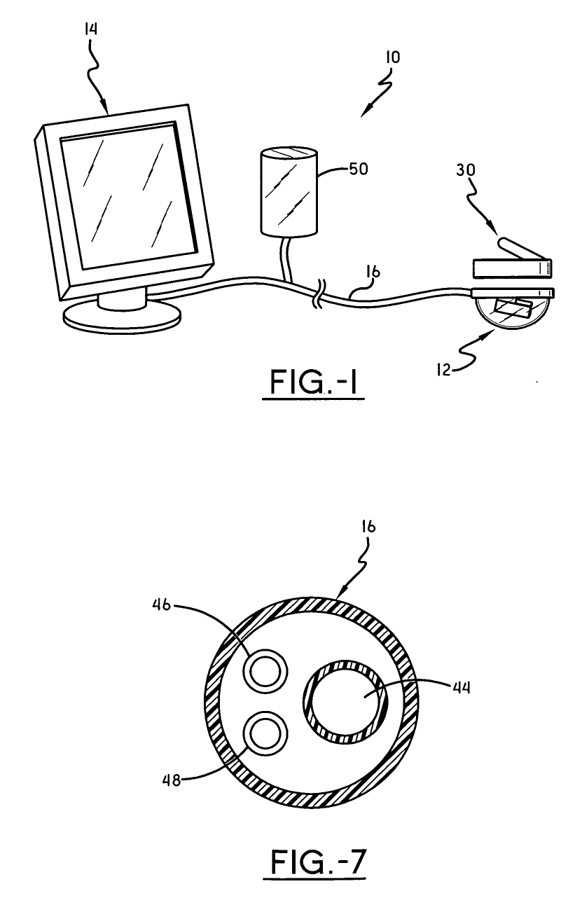

Referring now to FIG. 1, an embodiment of an in vivo imaging system in accordance with this invention is shown and designated by the numeral 10. Imaging system 10 includes a camera assembly 12, which is intended to be introduced to an in vivo site. Camera assembly 12 communicates with an image display device 14, through a cable 16 so that images received by the camera assembly 12 can be displayed. Cable 16 would also preferably provide power to the camera assembly and may provide other functionalities, as will be described below.

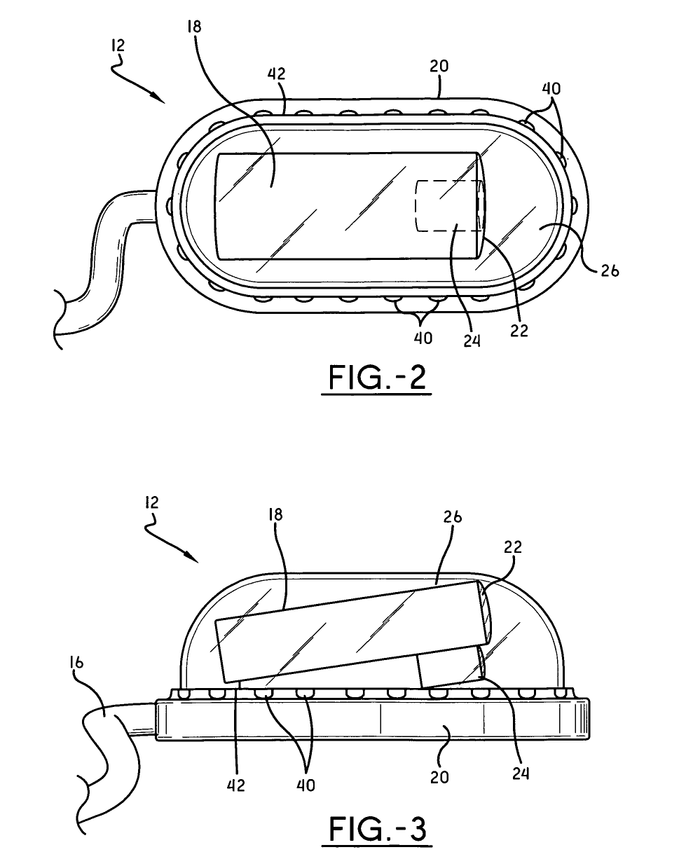

Referring now to FIGS. 2 and 3, camera assembly 12 includes a camera 18 that is secured to a sled body 20. Camera 18 includes a lens 22 having a viewing angle for taking in images and transmitting them to the image display device 14. As known, the viewing angle of the lens 22 could be made adjustable or fixed, with or without zoom capabilities and the like. Because the camera assembly 12 is targeted for placement in an in vivo site, a light 24 is also preferably provided mounted to either the sled body 20 or the camera 18 or some other structure on camera assembly 12. By providing a light as part of the assembly, a separate light source does not need to be introduced in vivo. A dome 26 is provided to cover and protect at least lens 22, though as shown here, dome 26 preferably covers both camera 18 and light 24. More particularly, in the embodiment shown, dome 26 is secured to sled body 20 and extends over both camera 18 and light 24 to define a sealed environment inside dome 26, between sled body 20 and dome 26.

Camera 18 is preferably similar to those types of cameras currently employed for taking images at an in vivo site. As such, it may be similar to endoscopes like those currently known and produced. Dome 26, sled body 20 and cable 16 should be made from suitable materials that are capable of being sterilized and are not harmful if introduced in vivo. Dome 26 should also be made such that camera 18 can receive images through the dome 26. In many embodiments, the dome 26 will be transparent, being made from glass or clear plastics, though it may be found that useful effects can be achieved by a tinted or otherwise suitably transparent dome 26. The sled body 20 is preferably made of steel or some other material capable of being held by a magnet as will now be described.

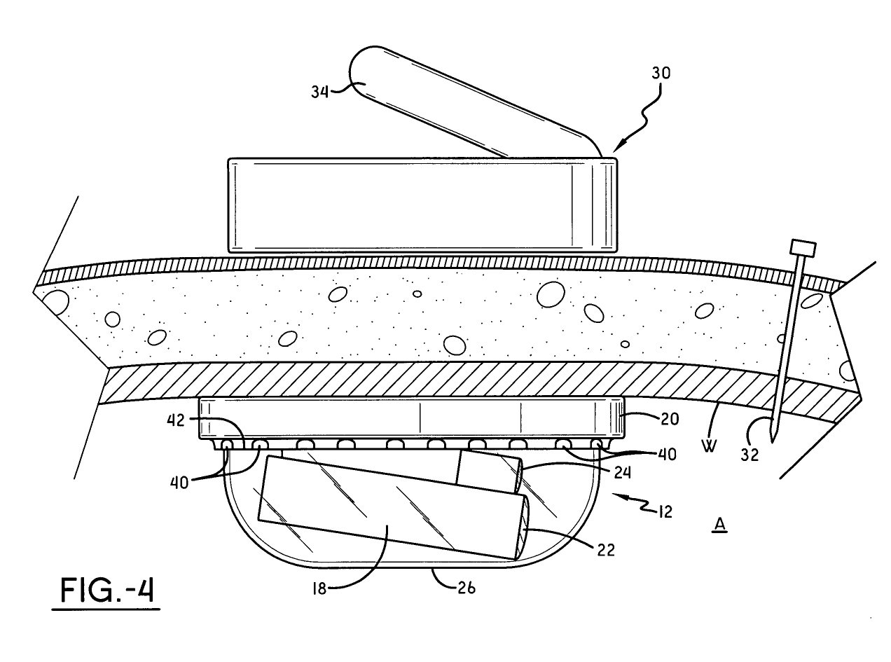

Particularly, sled body 20 is to be held in place at an in vivo site by a magnetic source body 30 placed on the exterior of the organism in which the camera assembly 12 is placed. In FIG. 4, the camera assembly 12 is placed inside the abdomen A of a patient, and the sled body 20 thereof is positioned against an internal sidewall W of the abdomen A.

The camera assembly is retained at this location by magnetic attraction of the sled body 20 by the magnetic source body 30. The force needed to hold the device will depend upon the distance between the magnetic source body 30 and the camera assembly 12, generally dictated by the mass of skin, muscle and adipose between the exterior of the organism and the internal location of the camera assembly 12. As is currently practiced, the abdomen A (or other organ or location in an organism) may be inflated as with carbon dioxide source 32, to raise the internal sidewall W from the remainder of the abdomen A and place the camera assembly at a raised location at which it is capable of being manipulated to view various procedures to take place in the abdomen A.

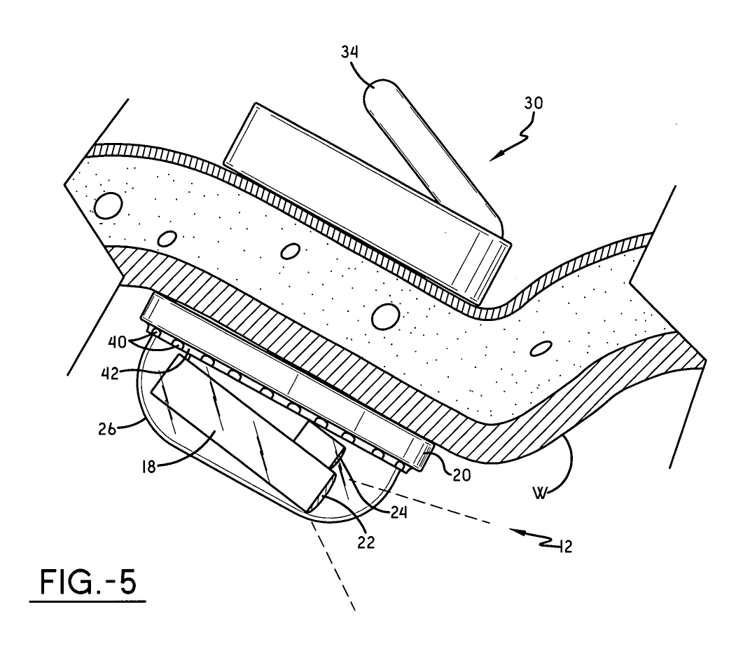

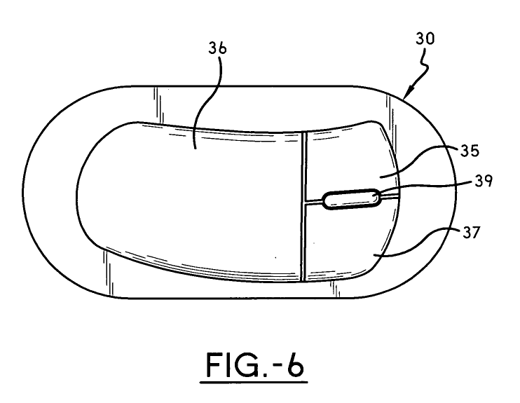

Once mounted at an in vivo site in this manner, the camera assembly 12 may be manipulated through movement of the magnetic source body 30 to provide a desired viewing angle. More particularly, because the skin, muscle and adipose tissue between the magnetic source body 30 and the sled body 20 are pliant, the magnetic source body 30 may be moved by direct manipulation at the exterior of the body, with such movement causing relative movement of the camera assembly 12. This is generally shown between FIGS. 4 and 5, where it can be seen that angling the magnetic source body A (as in FIG. 5) relative to a normal rest position (as in FIG. 4) causes the camera assembly 12 to also move. Grip 34 can be provided on the magnetic source body 30 for the purpose of providing an operator with the means for moving the camera assembly 12 in this manner. As seen in FIG. 6, such a grip could be provided in the general form of a common computer mouse 36, permitting a user-friendly means for manipulating the camera assembly 12, and, in particular embodiments, providing easy-to-use buttons 35, 37 and 39 for selected functions such as taking still pictures or zooming in and out or otherwise adjusting the viewing angle.

The sled body 20 is preferably smooth so that there is little friction between the sidewall W and the sled body 20 upon rotationally movement thereof through rotation of magnetic source body 30. Thus, magnetic source body 30 could be rotated, for example, from a 12 o'clock to a 6 o'clock position, to change the orientation of the sled body 20 (and thus the camera 18) by 180 degrees. In the embodiment of FIG. 6, magnetic source body 30 could be made to rotate relative to mouse 36, as indicated by motor 40 and axle 42, thus allowing a 360 degree rotation of the camera assembly 12, while maintaining the ergonomics of the mouse 36. That is, the camera assembly 12 could be rotated without requiring the operator of the mouse 36 to rotate the mouse 36.

The sled body 20 can be fed to the in vivo site in a number of ways, all currently known in the art. Incisions may be made through the skin and passage of a commercially available port through the adjacent tissues will allow the passage of the sled assembly into the in vivo site, as is common with laparoscopes and laparoscopic surgery instruments. When practical, the camera assembly 12 may also be fed to the in vivo site of interest through a natural orifice, for example, the mouth/esophagus or the vagina or rectum/colon. The cable 16 would extend out from the natural orifice or incision port, again as is common with laparoscopic practices. Removal of the camera assembly 12 after use will typically be in accordance with the placement, as is common in the laparoscopic arts.

It is believed that some magnetic source bodies could interfere with the electronics of the camera assembly 12, and, therefore, it is preferred to employ a DC current electromagnet. However, such may or may not be found to be necessary, and this invention is not limited to or by a particular magnetic source body.

During the course of using the camera assembly 12 at an in vivo site, the dome 26 may become smeared with blood or other bodily fluids or content, thereby compromising the image received from the camera 18 or the light emitted from light 24 or both. To address this concern, a plurality of rinse ports 40 are provided at the base 42 where dome 26 meets sled body 20. These rinse ports 40 communicate with a rinse tube 44 in cable 16 (or with a secondary cable, though having multiple cables would be less preferred). Thus, cable 16, as shown in FIG. 7, might carry rinse tube 44, power cable 46, and image communication cable 48. The invention is not limited to any particular manner for transmitting rise solution, power and images between the camera assembly and the solution source, power source or imaging system. As shown in FIG. 1, the cable may be split outside of the organism so that rinse tube 44 can communicate with a rinse solution source 50 so that a rinse solution such as water or saline can be fed to rinse ports 40. The rinse solution fed to the rinse ports 40 will tend to run in a sheet down over the dome 26 to clean the same.

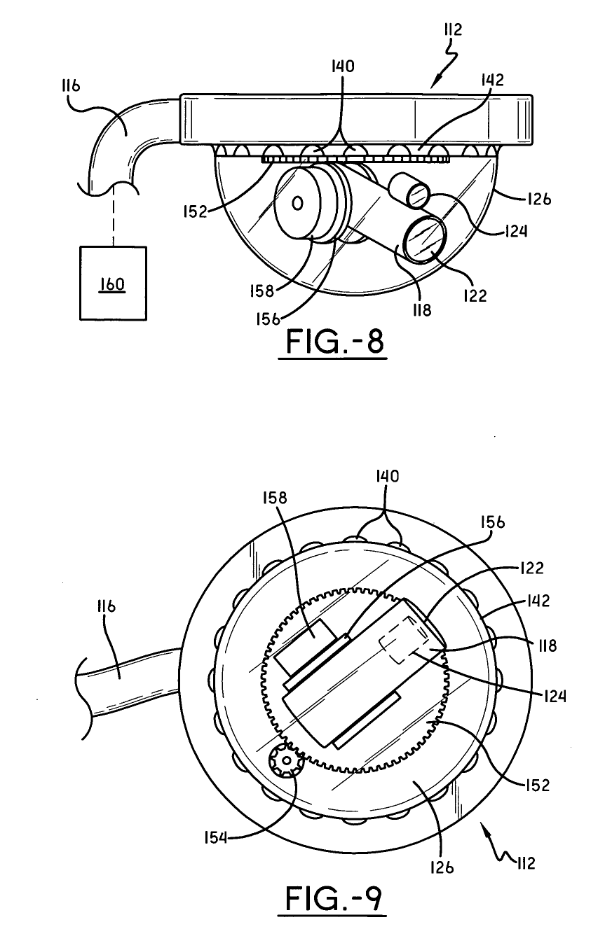

Another embodiment of a camera assembly is shown in FIGS. 8 and 9 and designated by the numeral 112. Camera assembly 112, which is intended to be introduced to an in vivo site, is powered and communicates with an image display device through cable 116, as generally described with respect to camera assembly 12 of imaging system 10. The camera assembly 112 includes a camera 118 that is secured to a sled body 120. Camera 118 includes a lens 122 having a viewing angle for taking in images and transmitting them to the image display device. As known, the viewing angle of the lens 122 could be made adjustable or fixed, with or without zoom capabilities and the like. Because the camera assembly 112 is targeted for placement in vivo, a light 124 is also preferably mounted to the camera 118 to move therewith. A dome 126 is provided to cover and protect at least lens 122, though as shown here, dome 126 preferably covers both camera 118 and light 124. More particularly, in the embodiment shown, dome 126 secures to sled body 120 and extends over both camera 118 and light 124 such that they reside in a sealed environment defined by sled body 120 and dome 126.

The sled body 120 is held in place at an in vivo site by an exterior magnetic source body as with the embodiment of imaging system 10. As with the mounting shown in FIG. 4, the camera assembly 112 would be placed inside the abdomen or other in vivo site of a patient, and the sled body 120 thereof would be secured to an internal sidewall by being magnetically pulled against an interior sidewall at the in vivo site. As already mentioned, the location in the organism may be inflated to raise the internal sidewall from the remainder of the body and place the camera assembly at a raised location at which it is capable of being manipulated to view various procedures.

Once mounted at an in vivo site, the camera assembly 112 may be manipulated either by manipulating the magnetic source body or through gearing to provide a desired viewing angle. More particularly, although capable of direct manipulation by movement of the magnetic source body 130, (as with the prior embodiment) camera 118 and light 124 are mounted to a rotating base 152 on sled body 120, and a rotating motor 154 can be actuated to rotate the rotating base 152 relative to sled body 120 to view in a complete 360 degree circle. Camera 118 and light 124 are also rotationally mounted to the rotating base 152, as at elevation wheel 156, actuated by elevation motor 158 to move the camera 118 in preferably a 180 degree arc from lying substantially parallel to rotating base 152, in one direction, to lying substantially parallel to rotating base 152, in the opposite direction, as shown in phantom in FIG. 8. Cable 116 would preferably provide the needed power, and would preferably also provide solution delivery to a plurality of rinse ports 140 provided at the base 142 where dome 126 meets sled body 120. A control system 160 would be provided for either wireless or wired control of the rotating motor 154 and elevation motor 158.

Although the power and image transmission of the imaging system herein have been disclosed as being transmitted through one or more cables, it should be appreciated that battery power and wireless image transmission are possible, and could make the use of cables unnecessary, except where a rinse solution is to be fed to irrigation ports, in which case a solution tube will be needed. However, there is no absolute requirement that the system include such irrigation ports, as there are other means for rinsing the dome of the camera assembly, as, for example, with a separate spray device introduced to the in vivo site.

In light of the foregoing, it should be apparent that the present invention improves the art by providing an in vivo imaging system that can be held out of the way of surgical instruments during surgery in vivo. While a particular embodiment of this invention has been the focus for purposes of disclosing the invention, it should be appreciated that this invention can be modified in various ways without departing from the general concepts taught herein. Thus, this invention is not to be limited to or by any particular embodiment, rather, the claims will serve to define the invention.

1. An in vivo imaging system for viewing the interior of an organism comprising:
a sled body placed in the interior of the organism;
a camera having a lens, said camera mounted to said sled body;
a dome covering said lens;
an image display device for displaying images received from said camera; and
a magnetic source body placed on the exterior of the body and magnetically attracting said sled body to hold said sled body in place in the interior of the organism.

2. The imaging system of claim 1, wherein said magnetic source body is manipulated to change the viewing angle of the lens.

3. The imaging system of claim 1, wherein said camera is mounted to said sled body to move to various viewing angles.

4. The imaging system of claim 3, wherein said camera is mounted to said sled body through a rotating base.

5. The imaging system at claim 4, wherein said camera is mounted to said rotating base through an elevation wheel.

6. The imaging system of claim 1, further including rinse ports to deliver a rinse solution to said dome, and a cable having a rinse tube for providing a rinse solution to said rinse ports.

7. The imaging system of claim 6, wherein said dome covers said camera and is secured to said sled body with said rinse ports located where said dome secures to said sled body.

8. The imaging system of claim 1, wherein said magnetic source body includes a grip for manipulating said magnetic source, said manipulation of said magnetic source serving to change the viewing angle of said lens.

9. The imaging system of claim 1, further including a cable communicating images to said image display device according to a viewing angle of the lens.

10. The imaging system of claim 1, including wireless transmission of images from said camera to said image display device.

11. A method of taking images at an in vivo site comprising the steps of:
positioning a camera assembly at an in vivo site, the camera assembly including:
a sled body,
a camera having a lens, the camera mounted to the sled body and receiving images in accordance with a viewing angle of the lens, and
a dome covering the lens;
securing the camera assembly at the in vivo site by aligning a magnetic source body with the sled body, the magnetic source body being positioned at an external, non-in vivo site and attracting the sled body; and
transmitting images received by the camera to an image display device for displaying the images.
